(19) United States
(12) Patent Application Publication
(10) Pub. No.: US 2019/0214138 A1
(43) Pub. Date: Jul. 11, 2019

(54) DIAGNOSIS SUPPORT APPARATUS, DIAGNOSIS SUPPORT SYSTEM, AND DIAGNOSIS SUPPORT METHOD

(71) Applicant: Canon Medical Systems Corporation, Otawara-shi (JP)

(72) Inventor: Kota AOYAGI, Nasushiobara (JP)

(73) Assignee: Canon Medical Systems Corporation, Otawara-shi (JP)

(21) Appl. No.: 16/237,774

(22) Filed: Jan. 2, 2019

(30) Foreign Application Priority Data
Jan. 10, 2018 (JP) 2018-002067
Dec. 26, 2018 (JP) 2018-242726

(51) Int. Cl. G16H 50/20; G16H 50/70; G16H 10/40; G16H 10/60; G16H 30/40

(52) U.S. Cl. CPC G16H 50/20; G16H 50/70; G16H 30/40; G16H 10/60; G16H 10/40

(57) ABSTRACT

According to one embodiment, a diagnosis support apparatus using a computation model includes processing circuitry. The processing circuitry generates an output signal by inputting feature quantities and supplementary values that substitute missing types of feature quantities. The processing circuitry inputs the supplementary values to the computation model while fluctuating the supplementary value. The processing circuitry selects at least one of the types of the feature quantities substituted by the supplementary values based on a change in value of an output signal. The processing circuitry generates support information based on the selected type of the feature quantity.

### CROSS-REFERENCE TO RELATED APPLICATIONS

This application is based upon and claims the benefit of priority from the prior Japanese Patent Application No. 2018-002067, filed Jan. 10, 2018 and No. 2018-242726, filed Dec. 26, 2018, the entire contents of both which are incorporated herein by reference.

### FIELD

Embodiments described herein relate generally to a diagnosis support apparatus, a diagnosis support system, and a diagnosis support method.

### BACKGROUND

By using machine learning information obtained from, for example, a medical image, and non-image information such as a result of a blood test and a smoking history, are used as an input to generate a trained model that realizes the inference of a disease name, inference of a malignancy degree of a disease, prognostic prediction, etc. Such trained model is used for a diagnosis support apparatus for the purpose of assisting a doctor in making a diagnosis, etc.

However, at a phase of actual use of a trained model at a medical site, all the pieces of data used to generate this particular trained model are not always prepared. In such a case, for example, a conceivable method is that a missing part of data is substituted by, for example, an average value in a population or a value inferred based on data regarding a patient. According to such a method, however, a result produced by the trained model becomes defective. In order to bring out the performance of the trained model, a missing piece of data needs to be complemented by performing an additional test, etc.; however, it is difficult to choose an optimal test.

### BRIEF DESCRIPTION OF THE DRAWINGS

FIG. 1 is a block diagram illustrating a functional configuration of a hospital information system provided with a diagnosis support apparatus according to a first embodiment.

FIG. 2 is a block diagram illustrating a functional configuration of the diagnosis support apparatus illustrated in FIG. 1.

FIG. 3 is a diagram illustrating a medical signal processing system that generates a trained model.

FIG. 4 is a diagram illustrating a configuration of a multi-layer network according to a present embodiment.

FIG. 5 is a flowchart illustrating operation at a time when processing circuitry executes inference processing using a trained model illustrated in FIG. 2.

FIG. 6 is a diagram explaining generation of a trained model illustrated in FIG. 2.

FIG. 7 is a diagram illustrating an input example of feature quantities to the trained model illustrated in FIG. 2.

FIG. 8 is a diagram illustrating a change in value of an output signal relative to a fluctuation in supplementary value regarding type D.

FIG. 9 is a diagram illustrating a change in value of an output signal relative to a fluctuation in supplementary value regarding type E.

FIG. 10 is a diagram illustrating another example of a change in value of an output signal relative to a fluctuation in supplementary value regarding type D.

FIG. 11 is a diagram illustrating another example of a change in value of an output signal relative to a fluctuation in supplementary value regarding type E.

FIG. 12 is a diagram illustrating another input example of feature quantities to the trained model.

FIG. 13 is a block diagram illustrating a functional configuration of a diagnosis support apparatus according to Modification 2 of a first embodiment.

FIG. 14 is a diagram at a time when supplementary values regarding all types are input to the trained model illustrated in FIG. 13.

FIG. 15 is a flowchart illustrating operation at a time when processing circuitry according to Modification 2 of the first embodiment executes inference processing.

FIG. 16 is a block diagram illustrating a configuration of an environment provided with a diagnosis support apparatus according to a second embodiment.

FIG. 17 is a block diagram illustrating a functional configuration of the diagnosis support apparatus illustrated in FIG. 13.

### DETAILED DESCRIPTION

In general, according to one embodiment, a diagnosis support apparatus that supports a diagnosis using a computation model generated by inferring an input of a preset number of types of feature quantities includes processing circuitry. The processing circuitry is configured to generate an output signal by inputting the input feature quantities and supplementary values that substitute missing types of feature quantities if types of input feature quantities are insufficient by two or more types relative to the preset number of types. The processing circuitry is configured to input the supplementary values to the computation model while fluctuating the supplementary value with respect to each of the missing types of the feature quantities. The processing circuitry is configured to select at least one of the types of the feature quantities substituted by the supplementary values based on a change in value of an output signal that is generated when each of the fluctuated supplementary values is input. The processing circuitry is configured to generate support information based on the selected type of the feature quantity.

Embodiments will be described below with reference to the drawings.

### First Embodiment

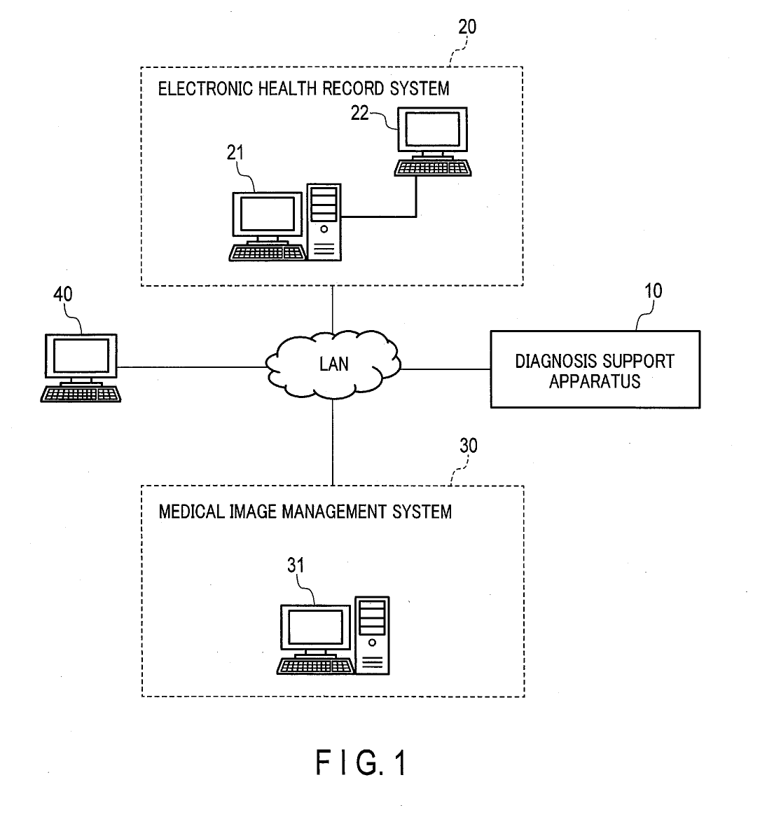

FIG. 1 is a block diagram illustrating a functional configuration of a hospital information system provided with a diagnosis support apparatus 10 according to a first embodiment. The hospital information system illustrated in FIG. 1 includes a diagnosis support apparatus 10, an electronic health record system 20, a medical image management system (PACS: Picture Archiving and Communication System) 30, and a communication terminal 40. The diagnosis support apparatus 10, the electronic health record system 20, the medical image management system 30, and the communication terminal 40 are connected through an intra-hospital network such as a LAN (Local Area Network) in a manner to enable data communication. It does not matter whether connection to an intra-hospital network is a wired connection or wireless connection. As long as security is ensured, a line to be connected is not limited to an intra-hospital network. For example, connection may be made to a public communication line such as to the Internet through a VPN (Virtual Private Network), etc.

The electronic health record system 20 is a system that stores electronic health record data including medical information, patient information, etc., and manages the stored electronic health record data. The medical information includes information on an electronic health record, such as finding information, disease name information, vital sign information, test stage information, and treatment content information. The patient information includes, for example, a patient ID, a patient's name, gender, age, etc.

The electronic health record system 20 includes, for example, a server apparatus 21 and a communication terminal 22. The server apparatus 21 and the communication terminal 22 are connected through an intra-hospital network in a manner to enable data communication. In the electronic health record system 20, the server apparatus 21 stores medical information, patient information, etc., and manages the stored medical information, patient information, etc. For example, the server apparatus 21 outputs the stored medical information, patient information, etc. to a requester in response to an output request.

FIG. 1 illustrates, as an example, the case in which a server included in the electronic health record system 20 is only the server apparatus 21. However, a server is not limited to this case. A plurality of server apparatuses 21 may be provided as needed. For example, the server apparatus 21 may be provided for each piece of information to manage.

The communication terminal 22 is a terminal for medical staff such as a doctor to access the server apparatus 21. Specifically, for example, the communication terminal 22 is operated by medical staff and requests the server apparatus 21 for information stored in the server apparatus 21.

The medical image management system 30 is a system that stores medical image data and manages the stored medical image data. The medical image management system 30 includes, for example, a server apparatus 31. Under the medical image management system 30, the server apparatus 31 stores medical image data converted in accordance with, for example, DICOM (Digital Imaging and Communication Medicine) standard, and manages the stored medical image data. For example, the server apparatus 31 transmits the stored medical image data to a requester in response to a browsing request.

FIG. 1 illustrates, as an example, the case in which the medical image management system 30 includes only the server apparatus 31. However, the server apparatus 31 is not limited to this case. A plurality of server apparatuses 31 may be provided as needed.

The communication terminal 40 is a terminal for medical staff such as a doctor to access a system, an apparatus, etc., which is connected to a LAN.

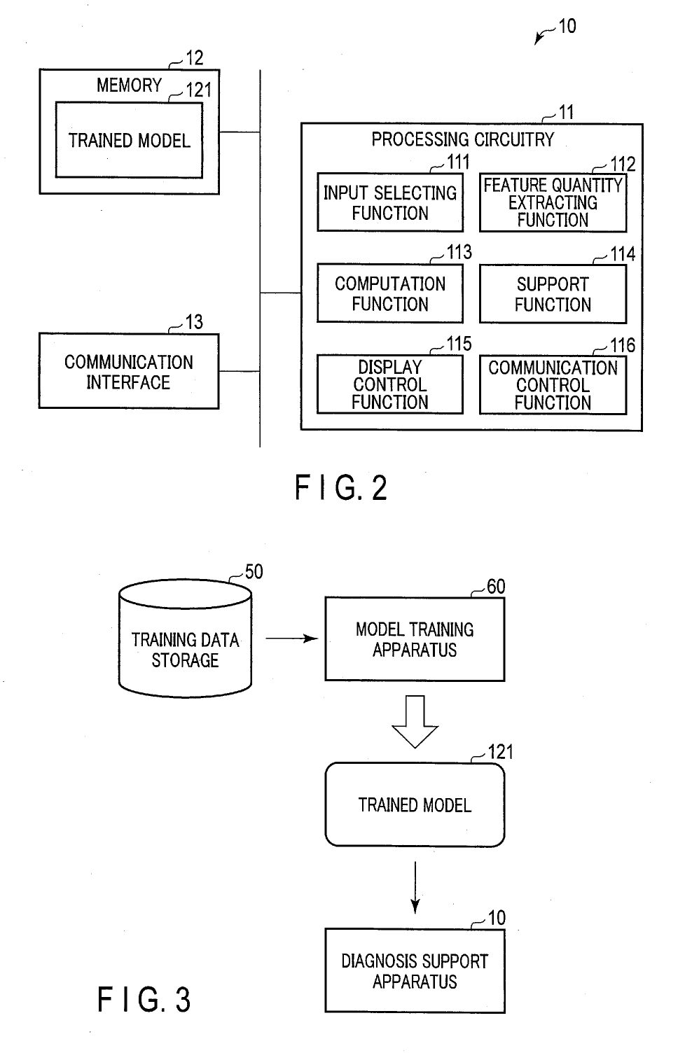

The diagnosis support apparatus 10 is an apparatus that supports an operator such as a doctor in diagnosing a patient. FIG. 2 is a block diagram illustrating an example of a functional configuration of the diagnosis support apparatus 10 illustrated in FIG. 1. The diagnosis support apparatus 10 illustrated in FIG. 2 includes processing circuitry 11, memory 12, and a communication interface 13. The processing circuitry 11, the memory 12, and the communication interface 13 are connected through, e.g., a bus so that they can communicate with each other.

The processing circuitry 11 is a processor that functions as a main unit of the diagnosis support apparatus 10. The processing circuitry 11 executes a program stored in the memory 12, etc., thereby realizing a function corresponding to this particular program.

The memory 12 is a storage device such as a ROM (Read Only Memory), a RAM (Random Access Memory), an HDD (Hard Disk Drive), an SSD (Solid State Drive), an integrated circuit storage device, etc., which stores a variety of information. The memory 12 may be a drive, etc., which reads and writes a variety of information with respect to a portable storage medium such as a CD-ROM drive, a DVD drive, a flash memory, etc. The memory 12 is not necessarily realized by a single storage device. For example, the memory 12 may be realized by a plurality of storage devices. Furthermore, the memory 12 may be located within another computer connected to the diagnosis support apparatus 10 through a network.

The memory 12 stores, a diagnosis support program, etc. according to the present embodiment. Such a diagnosis support program may be stored in advance in the memory 12, for example. Alternatively, such a diagnosis support program may be stored in a non-transitory storage medium and then distributed, thereby being read from the non-transitory storage medium and then installed in the memory 12. The memory 12, for example, stores a trained model 121 as an identification unit generated through machine learning. The trained model 121 is an example of a computation model. In the present embodiment, the trained model 121 represents a model that is generated by making a machine training model perform machine learning in accordance with a model training program.

The trained model 121 is generated as described hereinafter, for example, and is stored in the memory 12 of the diagnosis support apparatus 10.

FIG. 3 is a schematic diagram illustrating an example of a configuration of a medical signal processing system that generates the trained model 121. The medical signal processing system illustrated in FIG. 3 includes the diagnosis support apparatus 10, a training data storage 50, and a model training apparatus 60.

The training data storage 50 stores training data including a plurality of training samples. For example, the training data storage 50 is a computer having a mass-storage device incorporated therein. The training data storage 50 may be a mass-storage device that is connected to a computer through a cable or a communication network in a communicable manner. As this particular storage device, an HDD (Hard Disk Drive), an SSD (Solid State Drive), an integrated circuit storage device, etc., is available as appropriate.

The model training apparatus 60 generates the trained model 121 by making a machine training model perform machine learning in accordance with a model training program based on training data stored in the training data storage 50. In the present embodiment, examples of an algorithm for machine learning include discrimination analysis, logistic regression, a support vector machine, a neural network, Randomized Trees, a subspace method, etc. The model training apparatus 60 is a computer such as a workstation including a processor such as a CPU (Central Processing Unit) and GPU (Graphics Processing Unit).

The model training apparatus 60 and the training data storage 50 may be connected through a cable or a communication network so that they can communicate with each other. The training data storage 50 may be installed on the model training apparatus 60. In those cases, training data is supplied from the training data storage 50 to the model training apparatus 60. The model training apparatus 60 and the training data storage 50 may not be connected in a communicable manner. In such a case, training data is supplied from the training data storage 50 to the model training apparatus 60 through a portable storage medium having the training data stored therein.

The diagnosis support apparatus 10 and the model training apparatus 60 may be connected through a cable or a communication network so that they can communicate with each other. The diagnosis support apparatus 10 and the model training apparatus 60 may be installed in a single computer. In those cases, the trained model 121 generated by the model training apparatus 60 is supplied to the diagnosis support apparatus 10. The diagnosis support apparatus 10 and the model training apparatus 60 are not necessarily connected in a communicable manner. In such a case, the trained model 121 is supplied from the model training apparatus 60 to the diagnosis support apparatus 10 via a portable storage medium having the trained model 121 stored therein.

The trained model 121 may be supplied to the diagnosis support apparatus 10 at any point in time after the production of the diagnosis support apparatus 10. For example, the trained model 121 may be supplied to the diagnosis support apparatus 10 at any given point in time between the manufacture and the installation to a medical facility, etc., or at a time of maintenance. The trained model 121 which has been supplied is stored in the memory 12 of the diagnosis support apparatus 10.

The trained model 121 according to the present embodiment is a composite function with a parameter, which is prepared by combining a plurality of functions and is used to perform inference of a disease name, inference of a malignancy degree of a disease, or prognostic prediction, etc. by using a medical signal of medical image data and non-image medical information, etc., as inputs. A composite function with a parameter is defined by using the combination of adjustable functions and parameters. The trained model 121 according to the present embodiment may be any composite function with a parameter that satisfies the requirements described above.

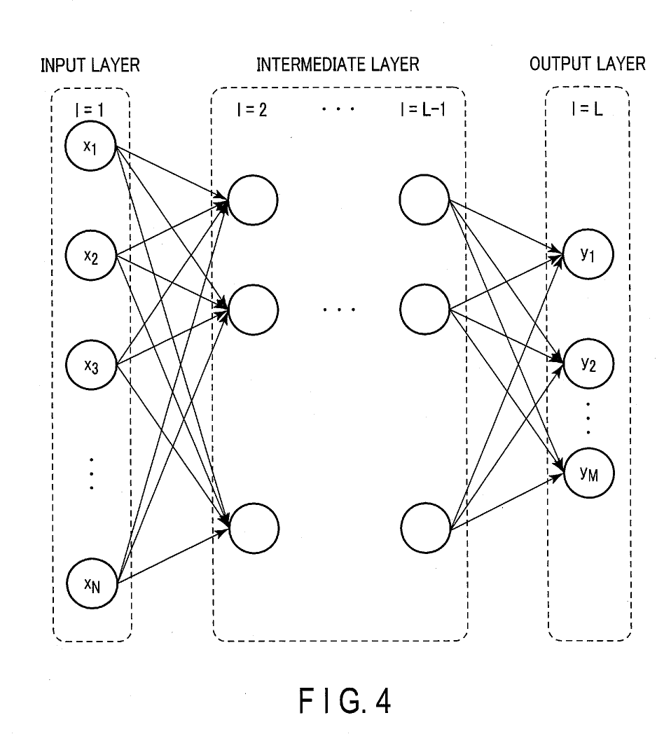

For example, the trained model 121 is generated using a multi-layer network of a sequent propagation type. The multi-layer network of the sequent propagation type according to the present embodiment is configured so that only adjacent ones of laminated layers are coupled to each other, as illustrated in FIG. 4, and information is propagated in one direction from an input-layer side to an output-layer side. The multi-layer network illustrated in FIG. 4 is composed of L layers including an input layer (I=1), an intermediate layer (I=2, 3, . . . , L−1), and an output layer (I=L).

If the trained model 121 is generated using the multi-layer network of the sequent propagation type, the composite function with a parameter is defined as, for example, a combination of a linear relationship between layers using a weight matrix, a nonlinear relationship (or linear relationship) using an active function in each layer, and a bias. As an activation function, various functions such as a logistic sigmoid function (logistic function), a hyperbolic tangent function, a linear normalization function (ReLU: Rectified Liner Unit), linear mapping, identity mapping, a maxout function, etc., depending on a purpose.

A weight matrix and bias is referred to as a parameter of a multi-layer network. A composite function with a parameter changes its form as a function, depending on how a parameter is selected. In the multi-layer network, appropriate setting of a constitutive parameter enables a function to be defined in a manner such that a preferable result is output from an output layer.

A parameter is set by execution of training using training data and an error function. For example, the training data is group D (n=1, . . . , S) of training samples (xₙ, dₙ) in which an input is predetermined input xₙ and a desirable outcome (correct output) with respect to this particular input is output dₙ. An error function is a function that presents the proximity between the correct output dₙ and an output from the multi-layer network in which Xₙ is input. Representative examples of an error function include a square error function, a maximum-likelihood estimation function, a crossover entropy function, etc. A function selected as an error function depends on a problem which the multi-layer network deals with (for example, a regression problem, a binary problem, a multi-class classification problem, etc.). A parameter is determined for each training sample. For example, a value that minimizes an error function is determined to be a parameter. In order to suppress the computation amount at a time of determining a parameter, an error backward propagation method may be used.

To be more specific, for example, the model training apparatus 60 according to the present embodiment performs machine learning based on training data in which an input is set to a predetermined feature quantity in medical image data and to a predetermined feature quantity in non-image data such as medical information, while a correct output is set to a diagnosed disease name. A predetermined feature quantity in medical image data is, for example, a texture feature quantity such as an average value, kurtosis, skewness, GLCM (Gray-Level Co-occurrence Matrix), etc. A predetermined feature quantity in non-image data is, for example, a result of a blood test, a smoking history, gender, and family information, a result of genomic analysis, etc. The model training apparatus 60 generates the trained model 121 that infers a disease name based on input feature quantities.

The model training apparatus 60 may generate the trained model 121 that infers a malignancy degree of a disease based on input feature quantities, by performing machine learning based on training data in which an input is set to a predetermined feature quantity in medical image data and to a predetermined feature quantity in non-image data such as medical information, while a correct output is set to a diagnostic outcome of a malignancy degree of a disease.

The model training apparatus 60 may generate the trained model 121 that performs prognostic prediction based on input feature quantities, by performing machine learning based on training data in which an input is set to a predetermined feature quantity in medical image data obtained at a plurality of points in time and to a predetermined feature quantity in non-image data such as medical information, while a correct output is set to a health condition after the lapse of a predetermined period from surgery.

The communication interface 13 illustrated in FIG. 2 performs data communication between the electronic health record system 20, the medical image management system 30, and the communication terminal 40, which are connected through an intra-hospital network. The communication interface 13 performs data communication in accordance with a known standard set in advance, for example. Communication with the electronic health record system 20 is performed in accordance with, e.g., HL7. Communication with the medical image management system 30 is performed in accordance with, e.g., DICOM.

The diagnosis support apparatus 10 may include an input interface. The input interface receives various types of operations input by a user, and converts the received input operation into an electrical signal, thereby outputting it to the processing circuitry 11. The input interface is connected to an input device such as a touch panel to which a command is input through contact with, for example, a mouse, a keyboard, a trackball, a switch, a button, a joystick, a touch-pad, an operation surface, etc. The input device connected to the input interface may be an input device provided in another computer connected through a network, etc.

The diagnosis support apparatus 10 may include a display. The display displays a variety of information in accordance with a command from the processing circuitry 11. The display may display, e.g., GUI (Graphical User Interface) for receiving various types of operation from a user. As the display, any display such as a CRT (Cathode Ray Tube) display, a liquid crystal display, an organic EL display, an LED display, a plasma display, etc., may be used as appropriate.

The processing circuitry 11 illustrated in FIG. 2 executes a diagnosis support program stored in the memory 12, thereby realizing a function corresponding to this program. For example, by execution of the diagnosis support program, the processing circuitry 11 includes an input selecting function 111, a feature quantity extracting function 112, a computation function 113, a support function 114, a display control function 115, and a communication control function 116. Described in the present embodiment is the case in which a single processor realizes the input selecting function 111, the feature quantity extracting function 112, the computation function 113, the support function 114, the display control function 115, and the communication control function 116. However, these functions are not limited to this example. For example, independent processors may be combined into the processing circuitry so that the input selecting function 111, the feature quantity extracting function 112, the computation function 113, the support function 114, the display control function 115, and the communication control function 116 are executed by the processors executing programs, respectively.

The input selecting function 111 is a function that selects, from data stored in a hospital information system, a medical signal to input to the trained model 121. Specifically, for example, by the input selecting function 111, the processing circuitry 11 selects data as a source from which a feature quantity set with respect to the trained model 121 is extracted. A medical signal to be selected is, for example, electric health record data stored in the electronic health record system 20 and image data in the medical image management system 30, such as ultrasonic image data, CT (Computed Tomography) image data, X-ray image data, MRI (Magnetic Resonance Imaging) image data, and PET (Positron Emission computed. Tomography) image data.

The feature quantity extracting function 112 is a function that extracts feature quantities from a read medical signal. Specifically, for example, by the feature quantity extracting function 112, the processing circuitry 11 extracts, from non-image data read from the electronic health record system 20, a plurality of types of feature quantities set with respect to the trained model 121, such as a result of blood test, a smoking history, gender, family, a result of genomic analysis, etc. The processing circuitry 11 extracts, for example, from image data read from the medical image management system 30, a plurality of types of feature quantities set with respect to the trained model 121, such as a texture feature quantity such as an average value, kurtosis, skewness, GLCM, etc.

The computation function 113 is a function that inputs extracted feature quantities to the trained model 121 and generates an output signal corresponding to the input feature quantities. Specifically, for example, by the computation function 113, the processing circuitry 11 inputs to the trained model 121, feature quantities of all types that are preset with respect to the trained model 121, and generates an output signal.

If there is a shortage of extracted types of feature quantities, the processing circuitry 11 sets a supplementary value to a feature quantity of a missing type. A supplementary value is a value used in substitution for a feature quantity of a missing type, and is preset for each type based on, e.g., an average or intermediate value of a population. A supplementary value may be set in advance based on, e.g., electronic health record data. The processing circuitry 11 inputs extracted feature quantities and supplementary values set with respect to each missing types of the feature quantities, to the trained model 121, thereby generating an output signal.

The support function 114 is a function that supports selection of a test to implement next if there is a shortage of feature quantities input to the trained model 121. Specifically, by the support function 114, the processing circuitry 11 fluctuates each of the supplementary values input to the trained model 121, in accordance with a predetermined rule. A predetermined rule is, for example, making an increment or a decrement within a predetermined range from a set supplementary value. At this point, an increment and a decrement may be made within a predetermined range with a set supplementary value being a center, or an increment or a decrement may be made with a set supplementary value being a start point.

The processing circuitry 11 analyzes a change in value of an output signal that is generated by inputting each fluctuation value to the trained model 121. The processing circuitry 11 generates support information based on the analysis result. For example, the processing circuitry 11 selects a type of a feature quantity which has a greater influence on the output signal between the insufficient types of feature quantities as support information.

Based on the selected type of a feature quantity, the processing circuitry 11 may obtain a test by which a feature quantity of the selected type can be obtained, and may include the obtained test in the support information.

The processing circuitry 11 may include in the support information a change in the output signal when a fluctuated supplementary value is input to the trained model 121. The processing circuitry 11 may include information on the test in the support information. For example, the information on a test includes, for example, at least one of a cost, the exposure amount, the presence or absence of invasiveness, and an implementable time when a test can be implemented. The processing circuitry 11 may include in the support information an evidence level of selecting a type of a feature quantity. Evidence levels are to grade a general tendency of the strength of conclusions according to research classification.

The display control function 115 is a function that controls display regarding output of the trained model 121. Specifically, for example, by the display control function 115, the processing circuitry 11 generates display data for use to display an output result generated by the computation function 113.

If a type of a feature quantity is selected by the support function 114, the processing circuitry 11 generates display data by adding support information generated by the support function 114 to the output result generated by the computation function 113.

The communication control function 116 is a function that controls communication between the electronic health record system 20, the medical image management system 30, and the communication terminal 40. Specifically, for example, by the communication control function 116, the processing circuitry 11 accesses the electronic health record system 20 and the medical image management system 30, and reads a medical signal selected using the input selecting function 111 from the electronic health record system 20 and the medical image management system 30. The processing circuitry 11 accesses the communication terminal 40 and transmits display data generated by the display control function 115 to the communication terminal 40 through a LAN.

Next, a diagnosis support operation by the diagnosis support apparatus 10 configured as described above will be described in accordance with a processing procedure of the processing circuitry 11.

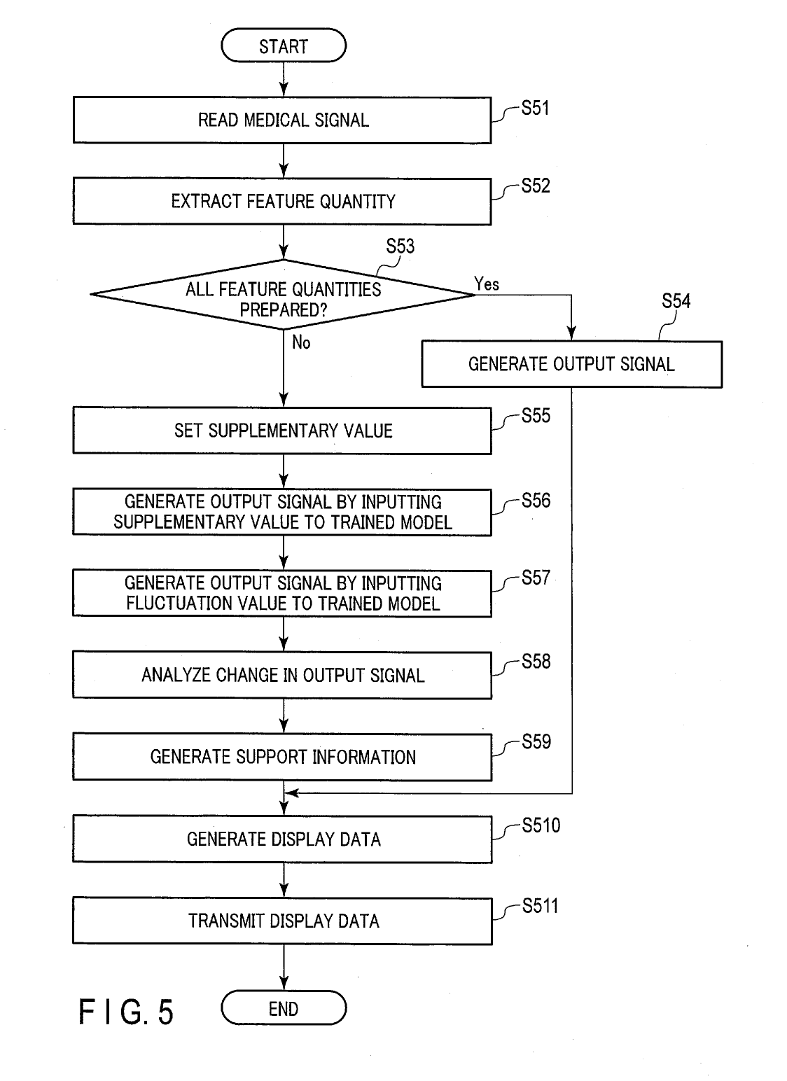

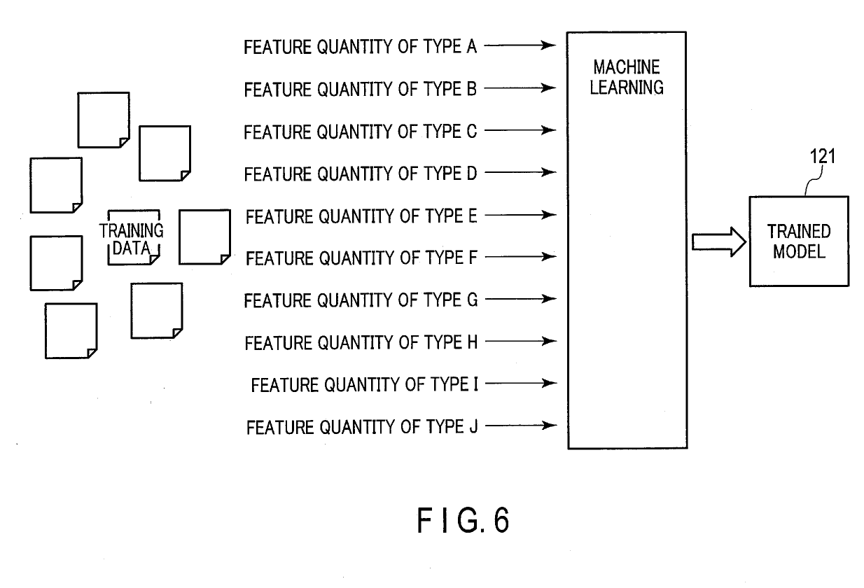

FIG. 5 is a flowchart illustrating an example of operation when the processing circuitry 11 of the diagnosis support apparatus 10 executes inference processing using the trained model 121. FIG. 5 illustrates, as an example, the case in which the trained model 121 is generated as illustrated in FIG. 6. That is, it is assumed that the trained model 121 is generated by performing machine learning based on training data in which an input is set to, for example, feature quantities of 10 types (A to J), while a correct output is set to a diagnostic outcome of a malignancy degree of a disease.

More specifically, it is assumed that the trained model 121 is a model to infer a malignancy degree of pulmonary nodule. A type of a feature quantity used to generate the trained model 121 is, for example, a size of pulmonary nodule in the case where a medical signal to be input is CT image data. A type of a feature quantity may be an average value of CT values of a pulmonary nodule. A type of a feature quantity may be a ratio (for example, a volume ratio) of a solid shadow to a ground-glass shadow. A type of a feature quantity may be a doubling time of pulmonary nodule. A type of a feature quantity may be a luminance distribution (for example, kurtosis and skewness) of pulmonary nodule. A type of a feature quantity may be GLCM. A type of a feature quantity is not limited to the above if a medical signal to be input is CT image data.

If a medical signal to be input is MRI image data, a type of a feature quantity may be a size of a pulmonary nodule in the MRI image data obtained through a predetermined sequence. A type of a feature quantity is not limited to the above if a medical signal to be input is MRI image data. If a medical signal to be input is PET image data, a type of a feature quantity may be a size of a region having a greater value of SUV (Standardized Uptake Value) in the PET image data. A type of a feature quantity is not limited to the above if a medical signal to be input is PET image data.

If a medical signal to be input is electric health record data, a type of a feature quantity may be, for example, a result of a blood test. A type of a feature quantity may be a smoking history. A type of a feature quantity may be gender. A type of a feature quantity may be family information. A type of a feature quantity may be a result of genomic analysis. A type of a feature quantity is not limited to the above if a medical signal to be input is electronic health record data.

When an operator of the communication terminal 40 inputs a command for starting inference processing of a malignancy degree of a disease with respect to a designated patient, the processing circuitry 11 of the diagnosis support apparatus 10 reads a diagnosis support program from the memory 12, and executes the read diagnosis support program. When the processing circuitry 11 executes the diagnosis support program, processing illustrated in FIG. 5 is started.

In FIG. 5, the processing circuitry 11 executes the input selecting function 111. Upon execution of the input selecting function 111, the processing circuitry 11 selects medical signals as sources of which A to J types of feature quantities set with respect to the trained model 121. Upon selection of the medical signals to obtain, the processing circuitry 11 executes the communication control function 116. Upon execution of the communication control function 116, the processing circuitry 11 accesses the medical image management system 30 and reads a part of the selected medical signals, e.g., medical image data, with respect to a designated patient from the medical image management system 30. The processing circuitry 11 accesses the electronic health record system 20 and reads other part of the selected medical signals, e.g., non-image data, with respect to a designated patient from the electronic health record system 20 (step S51).

Upon receipt of medical signals read from, e.g., the electronic health record system 20 and the medical image management system 30, the processing circuitry 11 executes the feature quantity extracting function 112. Upon execution of the feature quantity extracting function 112, the processing circuitry 11 extracts a plurality of types of feature quantities set with respect to the trained model 121, from the non-image data read from the electronic health record system 20. The processing circuitry 11 further extracts a plurality of types of feature quantities set with respect to the trained model 121, from the medical image data read from, e.g., the medical image management system 30 (step S52). For extraction of the feature quantities from the medical image data, existing processing such as outline extraction processing and pattern matching processing may be adopted.

Upon execution of feature quantities from, e.g., the read medical signal, the processing circuitry 11 executes the computation function 113. Upon execution of the computation function 113, the processing circuitry 11 determines whether feature quantities of all preset types have been prepared or not (step S53). If feature quantities of all types have been prepared (“Yes” in step S53), the processing circuitry 11 inputs the extracted feature quantities to the trained model 121, thereby generating an output signal (step S54). That is, the processing circuitry 11 outputs a malignancy degree of a pulmonary nodule while the feature quantities extracted from the medical image data and the electronic health record data are input. A malignancy degree of the pulmonary nodule is output as, for example, the probability of being a malignant pulmonary nodule.

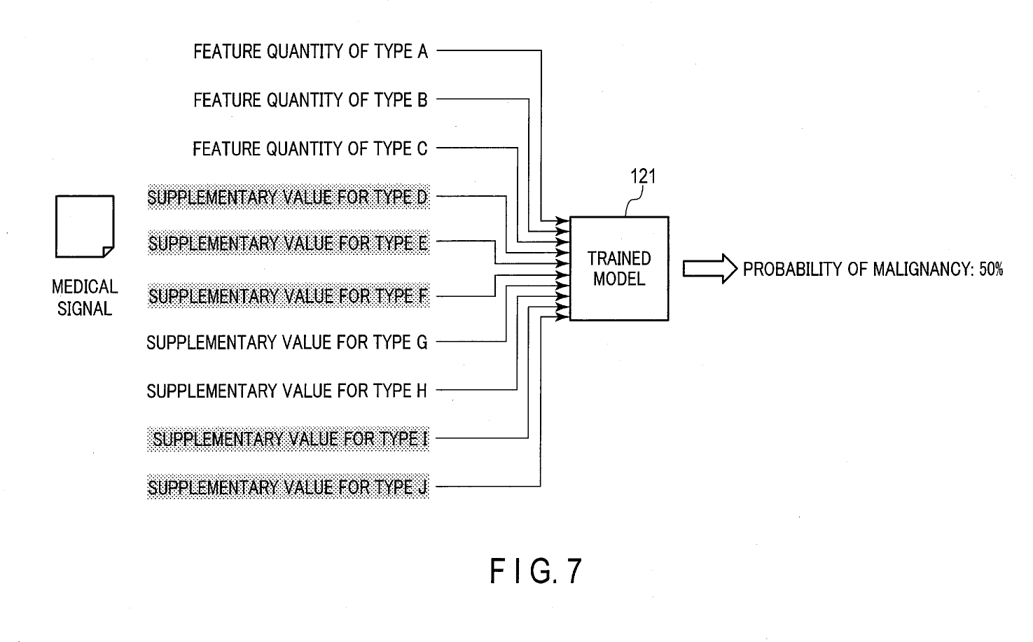

If the extracted types of feature quantities are insufficient relative to the preset types (“No” in step S53), the processing circuitry 11 sets supplementary values with respect to each missing types of feature quantities (step S55). For example, as illustrated in FIG. 7, regarding feature quantities of types A to J set in advance with respect to the trained model 121, assume that feature quantities of types A, B, C, G, and H are obtained while feature quantities of types D, E, F, I, and J are not obtained. In such a case, the processing circuitry 11 sets supplementary values that are assigned in advance with respect to types D, E, F, I, and J.

The processing circuitry 11 inputs the feature quantities extracted in step S52 and the supplementary values set in step S55 to the trained model 121, thereby generating an output signal (step S56). According to the example illustrated in FIG. 7, the processing circuitry 11 inputs the extracted feature quantities to the trained model 121 with respect to types A, B, C, G, and H, while inputting the supplementary values to the trained model 121 with respect to types D, E, F, I, and J. In this manner, the processing circuitry 11 outputs, for example, the 50-percent probability of being a malignant pulmonary nodule.

Upon execution of computation including supplementary-value computation, the processing circuitry 11 executes the support function 114. Upon execution of the support function 114, the processing circuitry 11 fluctuates one of the supplementary values input to the trained model 121 in accordance with a predetermined rule, thereby generating an output signal after the fluctuation of the supplementary value (step S57). Specifically, according to the example illustrated in FIG. 7, for example, the processing circuitry 11 changes the supplementary value with respect to type D between the supplementary values with respect to types D, E, F, I, and J by only the predetermined amount, and maintains preassigned supplementary values with respect to the other types, i.e., types E, F, I, and J. The processing circuitry 11 inputs to the trained model 121, the extracted feature quantities with respect to types A, B, C, G, and H, the fluctuated supplementary value with respect to type D, and the preassigned supplementary values with respect to types E, I, and J. In this manner, the processing circuitry 11 generates an output signal. The processing circuitry 11 repeatedly changes the supplementary value with respect to type D, and generates an output signal every time the supplementary value is changed.

Subsequently, the processing circuitry 11 changes the supplementary value with respect to type E between the supplementary values with respect to types D, E, F, I, and J, by only the predetermined amount, for example, and maintains preassigned supplementary values with respect to the other types, i.e., types D, F, I, and J. The processing circuitry 11 inputs to the trained model 121, the extracted feature quantities with respect to types A, B, C, G, and H, the fluctuated supplementary value with respect to type E, and the preassigned supplementary value with respect to types D, F, I, and J. In this manner, the processing circuitry 11 generates an output signal. The processing circuitry 11 repeatedly changes the supplementary value with respect to type E, and generates an output signal every time the supplementary value is changed. With respect to types F, I and J, the processing circuitry 11 repeats similar processing and generates output signals.

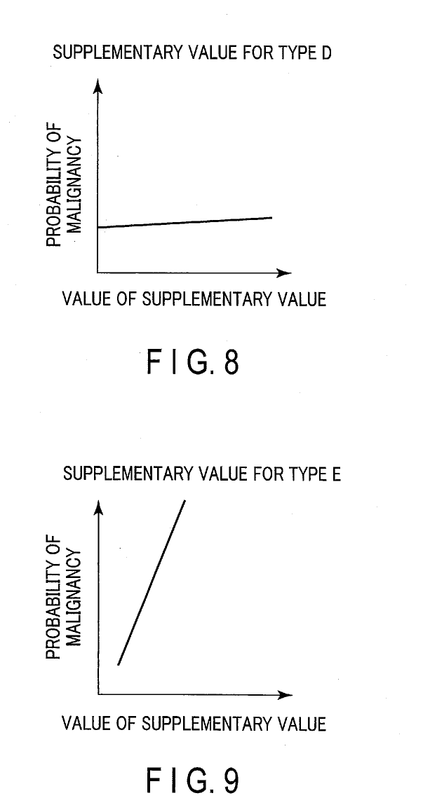

The processing circuitry 11 analyzes changes in value of the output signals generated by inputting each fluctuation value to the trained model 121 (step S58). Specifically, according to the example illustrated in FIG. 7, the processing circuitry 11 analyzes a change in value of the output signal generated when each of the fluctuated supplementary values for type D is input. FIG. 8 is a diagram illustrating an example of the change in value of the output signal relative to a fluctuation in the supplementary value with respect to type D. It is understood from FIG. 8 that even when the supplementary value for type D is fluctuated, the probability of being a malignant pulmonary nodule does not change much.

The processing circuitry 11 analyzes a change in value of the output signal that is generated when each of the fluctuated supplementary values for type E is input. FIG. 9 is a diagram illustrating an example of the change in value of the output signal relative to a fluctuation in the supplementary value with respect to type E. It is understood from FIG. 9 that when the supplementary value for type E is fluctuated, the probability of being a malignant pulmonary nodule changes significantly. With respect to types F, I and J, the processing circuitry 11 analyzes changes in value of the output signals in a similar manner.

The processing circuitry 11 generates support information in light of the analysis result regarding a change in value of the output signal (step S59). For example, the processing circuitry 11 compares the analysis result indicative of the tendency illustrated in FIG. 8 with the analysis result indicative of the tendency illustrated in FIG. 9, and selects type E which has a greater ratio of change in the probability of malignancy relative to the fluctuation in supplementary value, that is, has a highly-sensitized response, as a type which has a greater influence on the probability of being a malignant pulmonary nodule. More specifically, for example, type E corresponds to a size of a pulmonary nodule in MRI image data obtained through a predetermined sequence, and type D corresponds to a size of a region having a greater SUV value in PET image data. When a result of analyzing a change in value of an output signal, caused by fluctuating a supplementary value, shows that a size of a pulmonary nodule in MRI image data has a greater influence on the probability of being a malignant pulmonary nodule than a size of a region having a greater SUV value in PET image data, the processing circuitry 11 selects a size of the pulmonary nodule in MRI image data. The processing circuitry 11 generates support information including the selected type of a feature quantity.

A comparison made to select a dominant type is not limited to a comparison of ratio of change in the probability of malignancy relative to a fluctuation in supplementary value. Such a comparison may be made based on other indexes. For example, if values of output signals generated by fluctuating a supplementary value are varied beyond a preset range, the processing circuitry 11 may exclude the type corresponding to the supplementary value from a selection range.

The processing circuitry 11 may select a plurality of types that are determined to have a greater influence on an output signal. For example, when a ratio of change in the probability of malignancy relative to a fluctuation in supplementary value exceeds a preset value, corresponding types may be determined to have a greater influence on an output signal. In addition, the processing circuitry 11 may select a type of a feature quantity, which has the greatest influence on an output signal.

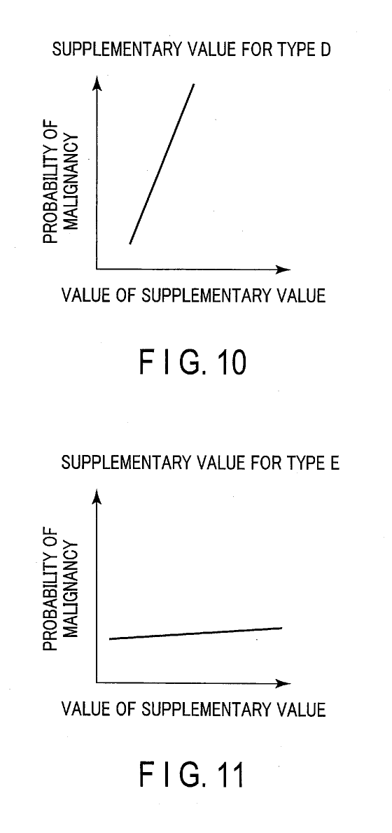

A type of a feature quantity, which is determined to have a greater influence on an output signal, depends on a relation with other feature quantities. Specifically, even a type of a feature quantity, which is determined to have a greater influence on an output signal with respect to patient A, does not always have an influence on an output signal with respect to patient B. That is, depending on a relation with other feature quantities, the change in value of the output signal with respect to type D is illustrated in FIG. 10, while the change in value of the output signal with respect to type E is illustrated in FIG. 11. In such a case, type D is selected as the type which has a greater influence on the probability of being a malignant pulmonary nodule.

Based on the selected type of a feature quantity, the processing circuitry 11 may further select a name of a test by which the selected type of a feature quantity can be obtained. This selection is based on the premise, for example, that the memory 12 stores a table in which types of feature quantities are associated with names of tests by which each types of feature quantities can be obtained. Based on the selected type of a feature quantity, the processing circuitry 11 reads a test by which this type of a feature quantity can be obtained, from the table stored in the memory 12. The processing circuitry 11 generates support information including the read test.

The processing circuitry 11 may include in the support information, the change in the output signal when each of the fluctuated supplementary values is input to the trained model 121. Specifically, for example, the change such as shown in FIGS. 8 to 11 is included in the support information.

The processing circuitry 11 may include a variety of information on the selected test in the support information. For example, the memory 12 stores a table in which a name of a test, by which a predetermined type of a feature quantity can be obtained, is associated with detailed information on this test. Detailed information on a test includes, for example, a scanning condition, a test cost, the presence or absence of invasiveness, etc. The scanning condition is, for example, a condition that is set for every scanning of the medical image diagnostic apparatus, and includes information on, for example, tube voltage, tube current, X-ray exposure time, etc. Based on the selected test, the processing circuitry 11 reads, from the table stored in the memory 12, detailed information on this test. Based on the read detailed information, the processing circuitry 11 generates support information including a cost, the exposure amount, the presence or absence of invasiveness, etc.

The processing circuitry 11 may include in the support information, an evidence level of selecting the type of a feature quantity. For example, the memory 12 stores a table in which a type of a feature quantity is associated with an evidence level of selecting this particular type. Based on the selected type of feature quantity, the processing circuitry 11 reads an evidence level regarding this particular type from the table stored in the memory 12. The processing circuitry 11 generates support information including the read evidence level.

The processing circuitry 11 may include an implementable time for the selected test in the support information. For example, an RIS (Radiological Information System), not shown, records implementation schedule information about an X-ray diagnostic apparatus, an X-ray CT apparatus, an MRI apparatus, an ultrasonic diagnostic apparatus, a SPECT (Single Photon Emission Computed Tomography) apparatus, a PET apparatus, an SPECT-CT apparatus having an SPECT apparatus and an X-ray CT apparatus integrated as one unit, a PET-CT apparatus having a PET apparatus and an X-ray CT apparatus integrated as one unit, a PET-MRI apparatus having a PET apparatus and an MRI apparatus integrated as one unit, etc. When a next test is selected, the processing circuitry 11 accesses the RIS. The processing circuitry 11 reads an implementable time when the desired test can be implemented, from implementation schedule information stored in the RIS. The processing circuitry 11 generates support information including the read implementable time.

For example, in step S54, when the output signal is generated by the trained model 121, the processing circuitry 11 executes the display control function 115. By the display control function 115, the processing circuitry 11 generates display data to display the output result generated in step S54 (step S510).

For example, in step S59, upon generating the support information, the processing circuitry 11 executes the display control function 115. By the display control function 115, the processing circuitry 11 generates display data for use to display the output result generated in step S56 and the support information generated in step S59 (step S510).

For example, upon generating the display data, the processing circuitry 11 executes the communication control function 116. Upon executing the communication control function 116, the processing circuitry 11 accesses the communication terminal 40 through a LAN, thereby transmitting the generated display data to the communication terminal 40 (step S511). The communication terminal 40 displays the received display data.

As described above, according to the first embodiment, if feature quantities extracted from an obtained medical image are insufficient in number relative to types of feature quantities inferred by the trained model 121, the diagnosis support apparatus 10 inputs supplementary values to the trained model 121 while fluctuating a supplementary value with respect to a missing feature quantity. The diagnosis support apparatus 10 selects a type of a feature quantity, which has a greater influence, based on a change in output value of the trained model 121, which is generated when each of the fluctuated supplementary values is input.

For example, the output of “50-percent probability of being a malignant pulmonary nodule” illustrated in FIG. 7 may be changed by replacing at least one of supplementary values with a true measured value. The output of a 70 percent probability indicates a large certainty factor of malignancy. In contrast, the output of a 30 percent probability indicates a small certainty factor of malignancy. Information with a large certainty factor is required for a doctor to make a diagnosis. Thus, if a certainty factor is not large enough, determination of a next test to implement is critical.

The diagnosis support apparatus 10 according to the first embodiment selects a feature quantity which contributes to a highly-sensitized change in output under imposed conditions (measured feature quantities and measured values of these feature quantities), by conducting a simulation, not by referring to an importance degree set in advance for feature quantities. This enables the diagnosis support apparatus 10 to present to an operator such as a doctor, what feature quantity to obtain next with respect to a designated patient.

Therefore, at a stage where a test is implemented, when an output signal is generated using input data obtained at this particular stage, it is possible to make a determination through a simulation regarding what feature quantity to input next in order to obtain useful information for diagnosis support.

The diagnosis support apparatus 10 according to the first embodiment includes in support information, a necessary test for extracting a feature quantity which gives a highly-sensitized change to an output. This enables the diagnosis support apparatus 10 to present a suitable test to implement next to an operator case by case.

The diagnosis support apparatus 10 according to the first embodiment includes in support information detailed information on a test by which a selected type of a feature quantity can be obtained. This enables an operator to discuss which test to implement next in consideration of a test cost, the exposure amount, the presence or absence of invasiveness, etc.

The diagnosis support apparatus 10 according to the first embodiment includes in support information an evidence level of selecting a type of a feature quantity. This enables an operator to discuss the correctness of a type of a feature quantity selected by the diagnosis support apparatus 10.

The diagnosis support apparatus 10 according to the first embodiment includes in support information an implementable time during which a selected test can be implemented. This enables an operator to determine a next test in consideration of time to implement the test.

Described as an example in the first embodiment is the case in which the diagnosis support apparatus 10 is connected to a LAN. However, the diagnosis support apparatus 10 is not limited to this case. The diagnosis support apparatus 10 may be housed in the electronic health record system 20. That is, the functions of the diagnosis support apparatus 10 may be implemented in the server apparatus 21 of the electronic health record system 20. The diagnosis support apparatus 10 being housed in the electronic health record system 20 enables a doctor to receive a suggestion for a next test to implement from the electronic health record system 20 while he or she diagnosing a patient.

(Modification 1)

Described as an example in the first embodiment is the case in which a type of a feature quantity is selected by referring to a change in value of an output signal generated by the single trained model 121. Modification 1 describes the case in which there are trained models, and a type of a feature quantity is selected by referring to a change in value in an output signal generated by each of the trained models.

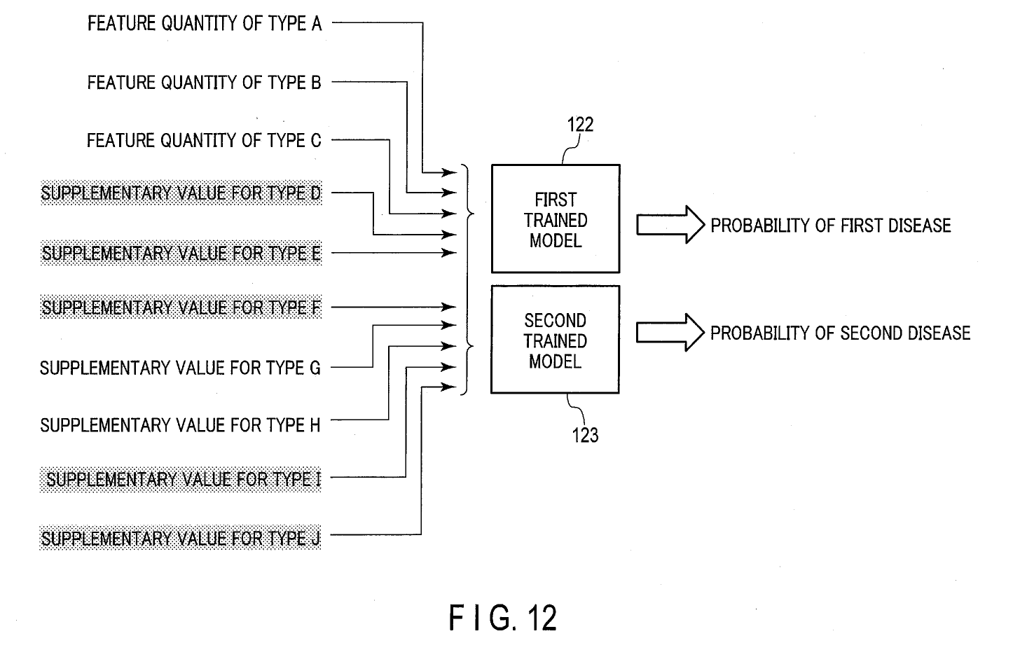

For example, as illustrated in FIG. 12, the memory 12 stores a first trained model 122 which infers a first disease based on input feature quantities, and a second trained model 123 which infers a second disease based on input feature quantities. FIG. 12 illustrates the case in which types of feature quantities input to the first trained model 122 are the same as types of feature quantities input to the second trained model 123. However, types of feature quantities to be input may be different for each trained model.

If extracted feature quantities are insufficient in number relative to the preset types, the processing circuitry 11 performs computation including a supplementary value in step S56 shown in FIG. 5. Subsequently, in step 57, the processing circuitry 11 fluctuates a supplementary value input to the first trained model 122 in accordance with a predetermined rule, thereby generating an output signal after the fluctuation of supplementary value. Furthermore, the processing circuitry 11 fluctuates a supplementary value input to the second trained model 123 in accordance with a predetermined rule, thereby generating an output signal after the fluctuation of supplementary value.

In step S58, the processing circuitry 11 analyzes a change in value of the output signal that is generated by inputting fluctuation values to the trained models 122 and 123, respectively. In step S59, the processing circuitry 11 compares the analysis results obtained in step S58. Specifically, between insufficient types of feature quantities, for example, the processing circuitry 11 selects a type of a feature quantity indicative of behavior of increasing an output of the first trained model 122, that is, the probability of the first disease, as well as decreasing an output of the second trained model 123, that is, the probability of the second disease as a type of a feature quantity which has a greater influence on the output signal. Alternatively, between insufficient types of feature quantities, for example, the processing circuitry 11 may select a type of a feature quantity indicative of behavior of decreasing an output of the first trained model 122, that is, the probability of the first disease, as well as increasing an output of the second trained model 123, that is, the probability of the second disease as a type of a feature quantity which has a greater influence on the output signal.

As described above, the diagnosis support apparatus 10 according to Modification 1 makes it possible to select a test to implement next, which is most suitable for differentiating between the first disease and the second disease, by a simulation under current conditions.

(Modification 2)

In the first embodiment, if any of types of feature quantities is missing, a supplementary value is set with respect to such a missing type, so that a type of a feature quantity, which has a greater influence, is selected by fluctuating the set supplementary value. In Modification 2, a type of a feature quantity, which is inferred to have a greater influence on a diagnosis, is set in advance. Described hereinafter is the case in which if any of types of feature quantities is missing with respect to a designated patient, first, a comparison is made with preset types of feature quantities and the missing type of a feature quantity to thereby select type of a feature quantity, which has a greater influence.

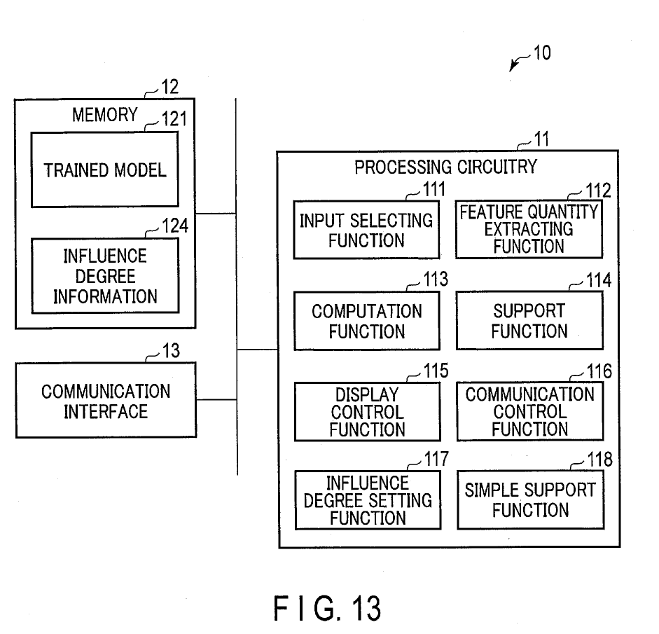

FIG. 13 is a block diagram illustrating the example of the functional configuration of the diagnosis support apparatus 10 according to Modification 2 of a first embodiment. The memory 12 illustrated in FIG. 13 stores not only the diagnosis support program according to the present embodiment, but also an influence degree setting program, etc., for example. The memory 12 stores not only the trained model 121, but also influence degree information 124, for example.

The processing circuitry 11 illustrated in FIG. 13 executes the diagnosis support program and the influence degree setting program stored in the memory 12, thereby realizing functions corresponding to these programs. By execution of the diagnosis support program and the influence degree setting program, the processing circuitry 11 includes the input selecting function 111, the feature quantity extracting function 112, the computation function 113, the support function 114, the display control function 115, the communication control function 116, an influence degree setting function 117, and a simple support function 118. Described in the present embodiment is the case in which a single processor realizes the input selecting function 111, the feature quantity extracting function 112, the computation function 113, the support function 114, the display control function 115, the communication control function 116, the influence degree setting function 117, and the simple support function 118. However, these functions are not limited to this example. For example, independent processors may be combined into the processing circuitry so that the input selecting function 111, the feature quantity extracting function 112, the computation function 113, the support function 114, the display control function 115, and the communication control function 116, the influence degree setting function 117, and the simple support function 118 are executed by the processors executing programs, respectively.

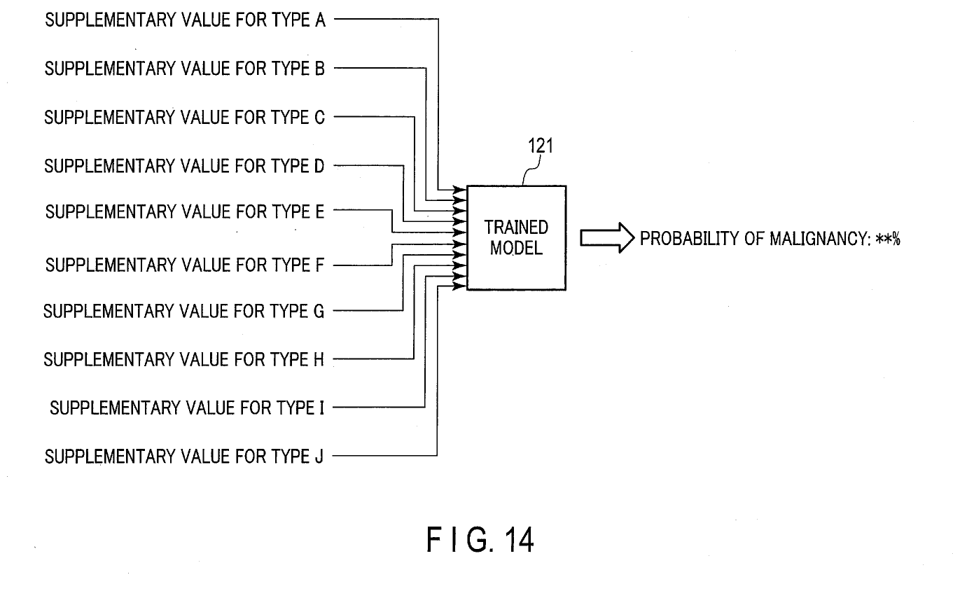

The influence degree setting function 117 is a function that sets a type of a feature quantity, which is inferred to have a greater influence on a diagnosis, with respect to the trained model 121 stored in the memory 12. Specifically, at a predetermined timing, for example, when the trained model 121 is stored in the memory 12, the processing circuitry 11 executes the influence degree setting program, thereby implementing the influence degree setting function 117. By the influence degree setting function 117, the processing circuitry 11 sets a supplementary value for each type, with respect to all types of feature quantities set with respect to the trained model 121. The processing circuitry 11 inputs all the set supplementary values to the trained model 121, thereby generating an output signal. FIG. 14 is a diagram at a time when supplementary values for all of the types are input to the trained model 121 in FIG. 13.

The processing circuitry 11 fluctuates any one of the supplementary values input to the trained model 121, in accordance with a predetermined rule. The processing circuitry 11 analyzes a change in value of an output signal that is generated by inputting each fluctuation value to the trained model 121. The processing circuitry 11 fluctuates all of the supplementary values by switching the supplementary values to fluctuate one by one. The processing circuitry 11 analyzes a change in value of an output signal output from the trained model 121 with respect to all the supplementary values. Based on the analysis results, the processing circuitry 11 sequences types of feature quantities in the order from the greatest influence on the output signal, for example. A great influence on the output signal indicates a great ratio of change in the output signal relative to the fluctuation in supplementary value, that is, a highly-sensitized response. The processing circuitry 11 sets influence degrees from 10 to 1 in the order from the greatest influence on the output signal, with respect to types A to J shown in FIG. 14. The processing circuitry 11 causes the memory 12 to store influence degrees set with respect to types A to J as the influence degree information 124 respectively.

The simple support function 118 is a function that supports selection of a test to implement next, based on the influence degree information 124 stored in the memory 12. Specifically, for example, by the simple support function 118, if extracted types of feature quantities are insufficient, the processing circuitry 11 collates a missing type of a feature quantity and the influence degree information 124, thereby generating support information based on a collation result. For example, the processing circuitry 11 selects a type of feature quantity which gives a greater influence on the output signal as support information between insufficient types of feature quantities.

Next, a diagnosis support operation by the diagnosis support apparatus 10 configured as described above will be described in accordance with a processing procedure of the processing circuitry 11.

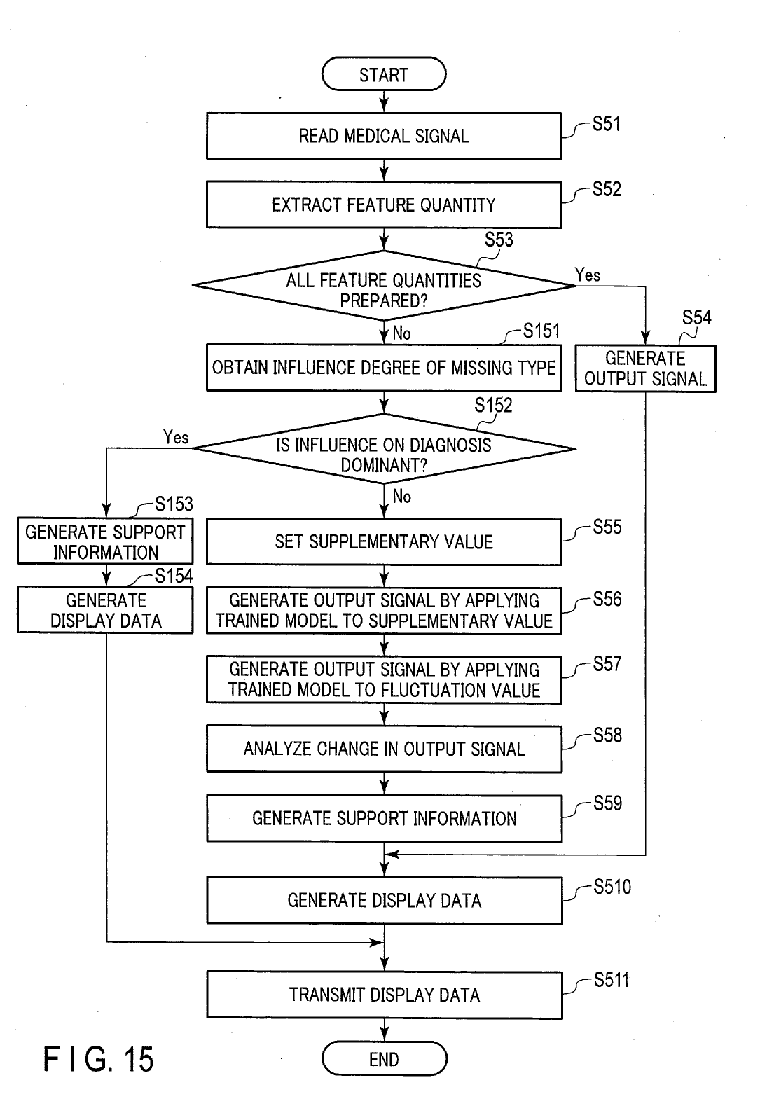

FIG. 15 is a flowchart illustrating an example of operation when the processing circuitry 11 of the diagnosis support apparatus 10 executes inference processing using the influence degree information 124 and the trained model 121. FIG. 15 illustrates, as an example, the case in which the trained model 121 is generated as illustrated in FIG. 6.

First, when an operator of the communication terminal 40 inputs a command for starting inference processing of a malignancy degree of a disease with respect to a designated patient, the processing circuitry 11 of the diagnosis support apparatus 10 reads a diagnosis support program from the memory 12, and executes the read diagnosis support program. When the processing circuitry 11 executes the diagnosis support program, processing illustrated in FIG. 15 is started.

In FIG. 15, the processing circuitry 11 implements processing from step S51 to step S53, which is the same as in FIG. 5, and determines whether feature quantities of all preset types have been prepared or not (step S53). If feature quantities of all types have been prepared (“Yes” in step S53), the processing circuitry 11 shifts processing to step S54. If extracted types of feature quantities are insufficient relative to the preset types (“No” in step S53), the processing circuitry 11 executes the simple support function 118. Upon execution of the simple support function 118, the processing circuitry 11 refers to the influence degree information 124 stored in the memory 12, thereby obtaining an influence degree with respect to a missing type of a feature quantity (S151).

Subsequently, the processing circuitry 11 determines whether or not a missing type of a feature quantity has a dominant influence on a diagnosis (step S152). An influence degree of a large value is set to a type of a feature quantity, which has the dominant influence on the diagnosis. Specific examples of the influence degree of the large value include the case of the influence degree taken as 8 to 10, the case of the influence degree taken as 9 and 10, the case of the influence degree taken as 10, etc. Specifically, for example, if feature quantities of types D, E, F, I, and J are missing, and influence degrees of 3, 4, 5, 6, and 10 are obtained for these types, respectively, type J, to which the influence degree of 10 is set, corresponds to a type of a feature that has the dominant influence on the diagnosis.

If a missing type of a feature quantity has the dominant influence on the diagnosis (“Yes” in step S152), the processing circuitry 11 generates support information using the missing type (step S153). Specifically, for example, the processing circuitry 11 generates support information including type J determined to have the dominant influence degree in step S152.

The processing circuitry 11 may select a plurality of types of feature quantities as being those having the dominant influence on the diagnosis. For example, if influence degrees of 9 and 10 are obtained for any missing type of a feature quantity, types for which influence degrees of 9 and 10 are obtained may be selected.

Based on the selected type of a feature quantity, the processing circuitry 11 may further select a name of a test by which the selected type of a feature quantity can be obtained. This selection is based on the premise, for example, that the memory 12 stores a table in which types of feature quantities are associated with names of tests by which each types of feature quantities can be obtained. Based on the selected type of a feature quantity, the processing circuitry 11 reads, from the table stored in the memory 12, a test by which this particular type of a feature quantity can be obtained. The processing circuitry 11 generates support information including the read test.

The processing circuitry 11 may include an influence degree obtained for a selected type in the support information. Specifically, for example, an influence degree of 10 for type J is included in the support information.

The processing circuitry 11 may include a variety of information on the selected test in the support information. For example, the memory 12 stores a table in which a name of a test by which a predetermined type of a feature quantity can be obtained is associated with detailed information on this test. Based on the selected test, the processing circuitry 11 reads detailed information on this particular test from the table stored in the memory 12. The processing circuitry 11 generates support information including information based on the read detailed information.

The processing circuitry 11 may include evidence information regarding the selected type of a feature quantity and the test in the support information. For example, the memory 12 stores a table in which a type of a feature quantity is associated with evidence information regarding a name of a test by which this type and a feature quantity of this type can be obtained. Based on the selected type of a feature quantity, the processing circuitry 11 reads evidence information regarding this particular type, from the table stored in the memory 12 based on the selected type of a feature quantity. The processing circuitry 11 generates support information including the read evidence information.

The processing circuitry 11 may include an implementable time for the selected test in the support information. When a next test is selected, the processing circuitry 11 reads an implementable time regarding the desired test from implementation schedule information stored in a predetermined server. The processing circuitry 11 generates support information including the read implementable time.

In step S153, for example, upon generating the support information, the processing circuitry 11 executes the display control function 115. By the display control function 115, the processing circuitry 11 generates display data to display the support information generated in step S153 (step S154). Upon generating the display data, the processing circuitry 11 shifts processing to step S511.

If there is no feature quantity of a type which has the dominant influence on the diagnosis (“No” in step S152), the processing circuitry 11 executes processing in step S55 to S511 by executing the computation function 113, the support function 114, etc.

As described above, with the diagnosis support apparatus 10 according to Modification 2, a type of a feature quantity, which has a dominant influence on a diagnosis, is checked in advance before a diagnosis. If a type of a feature quantity, which has a dominant influence on a diagnosis, is missing, the diagnosis support apparatus 10 supports a doctor in a manner so that a test to obtain a feature quantity of this missing type is implemented first. This enables diagnosis support to be efficiently performed.

### Second Embodiment

Described as an example in the first embodiment is the case in which the diagnosis support apparatus 10 is housed in a hospital information system. Described as an example in the second embodiment is the case in which a diagnosis support apparatus 10a is provided outside a hospital information system.

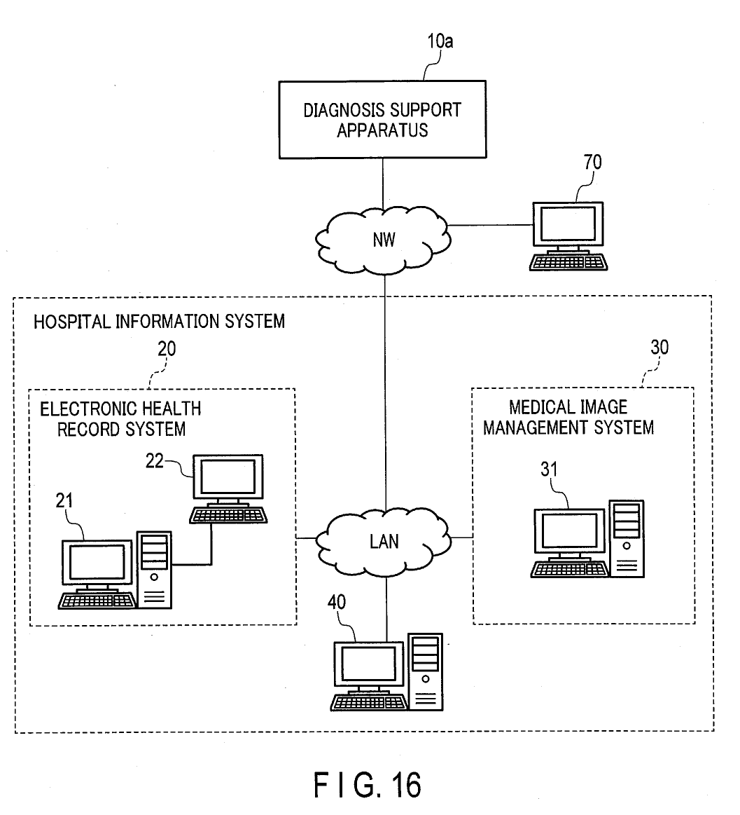

FIG. 16 is a block diagram illustrating a configuration of a diagnosis support system provided with the diagnosis support apparatus 10a according to the second embodiment. The diagnosis support apparatus 10a illustrated in FIG. 16 is connected to a hospital information system through a network such as the Internet, a communication net provided by a telecommunications carrier, etc. The diagnosis support apparatus 10a may be realized by a single server apparatus provided on a network, or may be realized by a plurality of server apparatuses.

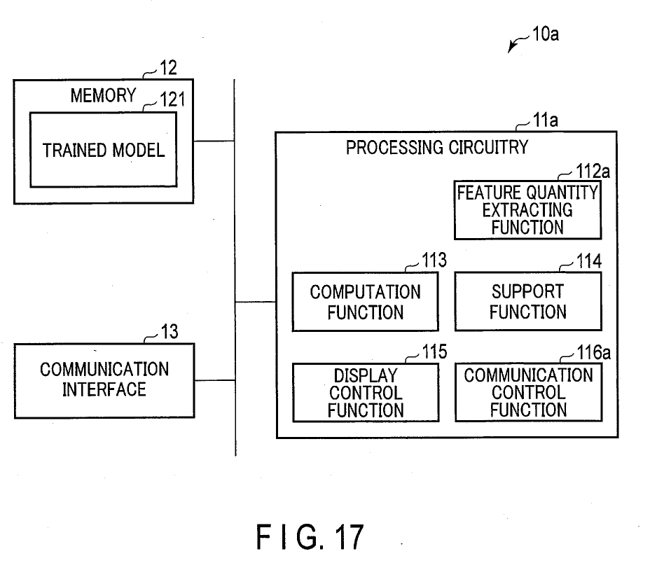

FIG. 17 is a block diagram illustrating an example of a functional configuration of the diagnosis support apparatus 10a illustrated in FIG. 16. The diagnosis support apparatus 10a illustrated in FIG. 17 includes processing circuitry 11a, the memory 12, and the communication interface 13. The processing circuitry 11a, the memory 12, and the communication interface 13 are connected through, e.g., a bus so that they can communicate with each other.

The processing circuitry 11a is a processor that functions as a main unit of the diagnosis support apparatus 10a. The processing circuitry 11a executes a diagnosis support program stored in the memory 12, etc., thereby realizing a function corresponding to the diagnosis support program. For example, by executing the diagnosis support program, the processing circuitry 11a includes a feature quantity extracting function 112a, the computation function 113, the support function 114, the display control function 115, and a communication control function 116a. Described in the present embodiment is the case in which a single processor realizes the feature quantity extracting function 112a, the computation function 113, the support function 114, the display control function 115, and the communication control function 116a. However, these functions are not limited to this example. For example, independent processors may be combined into the processing circuitry so that the feature quantity extracting function 112a, the computation function 113, the support function 114, the display control function 115, and the communication control function 116a are executed by the processors executing programs, respectively.

The feature quantity extracting function 112a is a function that extracts a feature quantity from a received medical signal. Specifically, for example, the diagnosis support apparatus 10a receives a request for diagnosis support from, e.g., the communication terminal 70 connected to a network, or the communication terminal 40 housed in a hospital information system. At this time, a request for diagnosis support includes medical image data and non-image data obtained by a test already implemented, in a condition that security is guaranteed. Upon receipt of a request for diagnosis support, the processing circuitry 11a of the diagnosis support apparatus 10a executes the feature quantity extracting function 112a. By the feature quantity extracting function 112a, the processing circuitry 11a extracts a plurality of types of feature quantities set with respect to the trained model 121, from the received medical image data and non-image data.

The communication control function 116a is a function that controls data communication through a network. Specifically, for example, by the communication control function 116a, the processing circuitry 11a is connected to the communication terminals 40 and 70 thorough a network and receives a request for diagnosis support. The processing circuitry 11a outputs an output result generated by the trained model 121 to the communication terminal 40 or 70 that had requested diagnosis support.

As described above, in the second embodiment, the diagnosis support apparatus 10a is provided on a so-called cloud. Upon receipt of a request for diagnosis support from a communication terminal connected to a network, the diagnosis support apparatus 10a inputs obtained feature quantities to the trained model 121, thereby outputting an output signal. If obtained feature quantities are insufficient in number relative to the types of feature quantities inferred by the trained model 121, the diagnosis support apparatus 10a inputs, while fluctuating a supplementary value with respect to a missing type of a feature quantity, the supplementary value to the trained model 121. The diagnosis support apparatus 10a selects a type of a feature quantity, which has a greater influence, based on a change in output value from the trained model 121, which is generated when each of the fluctuated supplementary values is input. This enables the diagnosis support apparatus 10a to present to an operator such as a doctor what feature quantity to obtain next with respect to a designated patient.

According to at least one of the embodiments described above, the diagnosis support apparatus is capable of supporting selection of an efficient additional test.

The term “processor” used in the above explanation of the embodiments means, for example, circuitry such as a CPU (Central Processing Unit) or a GPU (Graphics Processing Unit), an ASIC (Application Specific Integrated Circuit), a programmable logic device (for example, an SPLD (Simple Programmable Logic Device), a CPLD (Complex Programmable Logic Device), an FPGA (Field Programmable Gate Array), etc. A processor realizes its functions by reading and executing a program stored in storage circuitry. Instead of storing a program on storage circuitry, a program may be directly integrated into circuitry of a processor. In this case, a processor reads and executes a program integrated into circuitry, thereby realizing its functions. Each processor of the above embodiments is not limited to a configuration as a single circuit; and a plurality of independent circuits may be combined into one processor to realize its functions. Furthermore, a plurality of constituent elements in the above embodiments may be integrated into one processor to realize their functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A diagnosis support apparatus that supports a diagnosis using a computation model generated by inferring an input of a preset number of types of feature quantities, the diagnosis support apparatus comprising:
processing circuitry configured to:
if types of input feature quantities are insufficient by two or more types relative to the preset number of types, generate an output signal by inputting the input feature quantities and supplementary values that substitute missing types of feature quantities; and
input, while fluctuating the supplementary value with respect to each of the missing types of the feature quantities, the supplementary values to the computation model, select at least one of the types of the feature quantities substituted by the supplementary values based on a change in value of an output signal that is generated when each of the fluctuated supplementary values is input, and generate support information based on the selected type of the feature quantity.

2. The diagnosis support apparatus according to claim 1, wherein the processing circuitry is configured to:
while fluctuating each of supplementary values that substitute the preset number of types of the feature quantities respectively, input the supplementary values to the computation model, and set an influence degree to each of the preset number of types of the feature quantities, based on a change in value of an output signal that is generated when each of the fluctuated supplementary values is input; and
if types of input feature quantities are insufficient by two or more types relative to the preset number of types, determine whether or not any of the missing types of the feature quantities has a dominant influence, based on the influence degree, if any of the missing types of the feature quantities is determined to have a dominant influence, generate support information based on the missing type having the dominant influence, and if none of the missing types of the feature quantities is determined to have the dominant influence, input the input feature quantities and the supplementary values that substitute the missing types of the feature quantities to the computation model.

3. The diagnosis support apparatus according to claim 1, wherein the processing circuitry includes in the support information, a name of a test by which the selected type of the feature quantity is obtainable.

4. The diagnosis support apparatus according to claim 1, wherein the processing circuitry includes in the support information, the change in value of the output signal generated when each of the fluctuated supplementary values is input.

5. The diagnosis support apparatus according to claim 1, wherein the processing circuitry includes in the support information, at least one of a cost, an exposure amount, a presence or absence of invasiveness, and an implementable time with respect to a test by which the selected type of the feature quantity is obtainable.

6. The diagnosis support apparatus according to claim 1, wherein the processing circuitry includes in the support information, an evidence level of the selecting the type of the feature quantity.

7. The diagnosis support apparatus according to claim 1, wherein the processing circuitry generates display data including the output signal and the support information.

8. The diagnosis support apparatus according to claim 1, wherein the computation model is generated by machine learning.

9. A diagnosis support system that supports a diagnosis using a computation model generated by inferring an input of a preset number of types of feature quantities, the diagnosis support system comprising:
processing circuitry configured to:
if types of input feature quantities are insufficient by two or more types relative to the preset number of types, generate an output signal by inputting the input feature quantities and supplementary values that substitute missing types of feature quantities; and
input, while fluctuating the supplementary value with respect to each of the missing types of the feature quantities, the supplementary values to the computation model, select at least one of the types of the feature quantities substituted by the supplementary values based on a change in value of an output signal that is generated when each of the fluctuated supplementary values is input to the computation model, and generate support information based on the selected type of the feature quantity.

10. The diagnosis support system according to claim 9, wherein the processing circuitry is configured to:
input, while fluctuating each of supplementary values that substitute the preset number of types of the feature quantities respectively, the supplementary values to the computation model, and set an influence degree to each of the preset number of types of the feature quantities based on a change in value of an output signal that is generated when each of the fluctuated supplementary values is input; and
if types of input feature quantities are insufficient by two or more types relative to the preset number of types, determine whether or not any of the missing types of the feature quantities has a dominant influence, based on the influence degree, if any of the missing types of the feature quantities is determined to have a dominant influence, generate support information based on the missing type having the dominant influence, and if none of the missing types of the feature quantities is determined to have the dominant influence, input the input feature quantities and the supplementary values that substitute the missing types of the feature quantities to the computation model.

11. A diagnosis support method, comprising:
extracting a plurality of types of feature quantities from a medical signal;
comparing numbers between types of feature quantities inferred to be input to a computation model and the extracted types of the feature quantities;
if types of the extracted feature quantities are insufficient by two or more types relative to the inferred number of types, generating an output signal by inputting the extracted feature quantities and a supplementary values that substitute missing types of feature quantities;
fluctuating the supplementary value with respect to each of the missing types of the feature quantities, and generating an output signal by inputting the fluctuated supplementary value to the computation model; and
selecting at least one of the types of the feature quantities substituted by the supplementary values based on a change in value of an output signal generated when each of the fluctuated supplementary values is input.

12. The diagnosis support method according to claim 11, further comprising:
if types of input feature quantities are insufficient by two or more types relative to the present number of types, determining whether or not any of the missing types of the feature quantities has a dominant influence, based on an influence degree to each of the preset number of types of the feature quantities;
if any of the missing types of the feature quantities is determined to have a dominant influence, generating support information based on the missing type having the dominant influence; and
if none of the missing types of the feature quantities is determined to have the dominant influence, inputting the extracted feature quantities and a supplementary values that substitute missing types of feature quantities to the computation model.
